(19) United States
(12) Patent Application Publication
(10) Pub. No.: US 2020/0023150 A1
(43) Pub. Date: Jan. 23, 2020

(54) Airway Support Storage Platform

(71) Applicant: Horace Watson Barnett, Washington, DC (US)

(72) Inventor: Horace Watson Barnett, Washington, DC (US)

(21) Appl. No.: 16/508,841

(22) Filed: Jul. 11, 2019

Related U.S. Application Data
(60) Provisional application No. 62/699,811, filed on Jul. 18, 2018.

(51) Int. Cl. A61M 16/00; A61M 16/08

(52) U.S. Cl. CPC A61M 16/0084; A61M 16/0078; A61M 1/0039; A61M 16/0081; A61M 16/0875

(57) ABSTRACT

An airway support storage platform. The airway support storage platform includes a plate having a front side opposite a rear side, wherein a fastener is disposed on the rear side that can removably secure the plate to a support surface. A first adapter is affixed to the front side, wherein the first adapter includes an endotracheal tube connector. A second adapter is affixed to the front side, wherein the second adapter can frictionally engage an interior wall of a suction tubing thereon.

### CROSS REFERENCE TO RELATED APPLICATIONS

This application claims the benefit of U.S. Provisional Application No. 62/699,811 filed on Jul. 18, 2018. The above identified patent application is herein incorporated by reference in its entirety to provide continuity of disclosure.

### BACKGROUND OF THE INVENTION

The present invention relates to airway support storage platforms. More particularly, the present invention pertains to an airway support storage platform including a first and second adapter affixed to a plate, wherein the first and second adapter are configured to removably secure a manual resuscitation bag valve system and a suction tubing thereto, respectively.

Many hospitals and clinics require manual resuscitation bags and suction tubing to treat various ailments, however organizing and ensuring that such tools are readily available to personnel in times of emergency or otherwise can be difficult. Should a health professional be unable to retrieve a manual resuscitation bag or suction tubing in quick order, significant harm could be inflicted on a patient due to lack of airflow, such as brain damage or even death. Additionally, during emergency situations, such equipment is prone to being dropped or otherwise mishandled during retrieval and transport, which can quickly lead to contamination, leading to unsafe conditions for the patient. Therefore, a device that allows a user to store manual resuscitation bags and suction tubing adjacent to a patient treatment location for quick and efficient retrieval is desired.

In light of the devices disclosed in the known art, it is submitted that the present invention substantially diverges in design elements from the known art and consequently it is clear that there is a need in the art for an improvement to existing airway support storage platforms. In this regard, the instant invention substantially fulfills these needs.

### SUMMARY OF THE INVENTION

In view of the foregoing disadvantages inherent in the known types of airway support storage platforms now present in the known art, the present invention provides an airway support storage platform wherein the same can be utilized for providing convenience for the user when storing a manual resuscitation bag valve system and suction tubing for a specific patient adjacent to that patient.

The present system comprises a plate having a front side opposite a rear side, wherein a fastener is disposed on the rear side. The fastener is configured to removably secure the plate to a support surface. A first adapter is affixed to the front side, wherein the first adapter comprises an endotracheal tube connector. A second adapter is affixed to the front side, wherein the second adapter is configured to frictionally engage an interior wall of a suction tubing thereon. In some embodiments, the second adapter further comprises a plurality of ridges along an outer surface thereof. In another embodiment, the plurality of ridges are concentric. In other embodiments, the second adapter tapers from a proximal end thereof towards a distal end thereof, such that the distal end comprise a diameter less than that of the proximal end. In yet another embodiment, the first adapter is removably securable to the front side via a snap fit connection. In some embodiments, the second adapter is removably securable to the front side via a snap fit connection. In other embodiments, the fastener comprises a hook and loop fastener configured to removably secure to a complementary fastener disposed on the support surface. In another embodiment, the fastener is disposed along an entirety of the rear side of the plate, such that a perimeter of the fastener and a perimeter of the base are aligned. In yet another embodiment, an area of the fastener is less than that of the rear side, defining a lip about the rear side. In some embodiments, the fastener comprises an adhesive.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows an exploded view of an embodiment of the airway support storage platform with manual resuscitation bag valve system and suction tubing.

FIG. 2 shows a perspective view of the first adapter of an embodiment of the airway support storage platform.

FIG. 3 shows a perspective view of the second adapter of an embodiment of the airway support storage platform.

FIG. 4 shows a rear view of an embodiment of the airway support storage platform.

### DETAILED DESCRIPTION OF THE INVENTION

Reference is made herein to the attached drawings. Like reference numerals are used throughout the drawings to depict like or similar elements of the airway support storage platform. The figures are intended for representative purposes only and should not be considered to be limiting in any respect.

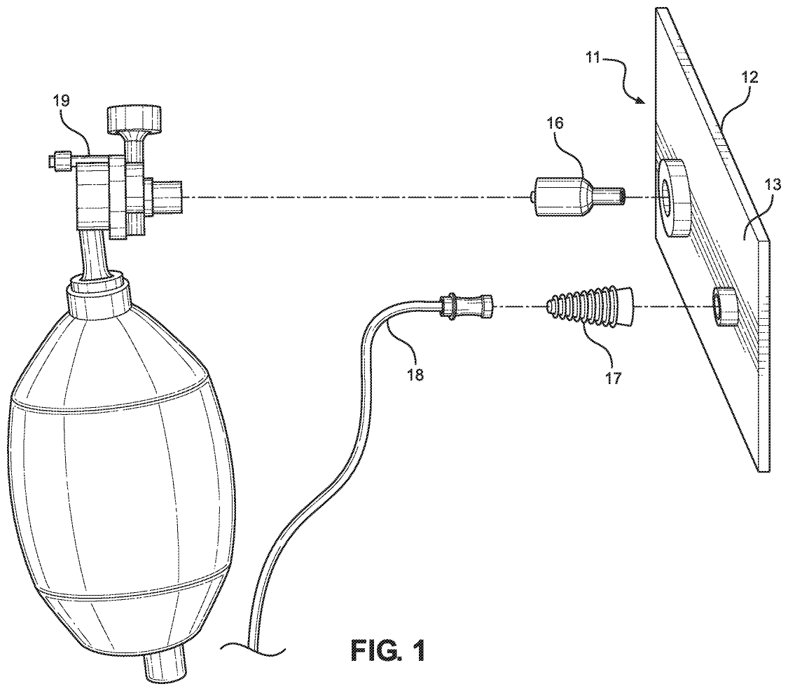

Referring now to FIG. 1, there is shown an exploded view of an embodiment of the airway support storage platform with manual resuscitation bag valve system and suction tubing. The airway support storage platform 11 comprises a plate 12 having a front side 13 opposite a rear side (as shown in FIG. 4, 14). A first adapter 16 and a second adapter 17 are each capable of securement to the front side 13, such that the first and second adapters 16, 17 are affixed thereto. In some embodiments, the first and second adapters 16, 17 are removably securable to the front side 13, such that a user can easily exchange the first and second adapters 16, 17 for alternate adapters of a desired type or size, while in alternate embodiments, the first and second adapters 16, 17 are permanently affixed to the front side 13. In the illustrated embodiment, the plate 12 further comprises a pair of connection points disposed on the front side 13, the pair of connection points configured to removably secure each of the first and second adapters 16, 17 therein. In some embodiments, the first and second adapters 16,17 are removably securable to the plate 12 via friction fit, while in other embodiments, the first and second adapters 16, 17 are secured via a locking mechanism, such as a ball detent or the like, to prevent the first and second adapters 16, 17 from being removed from the plate 12 when objects secured to the adapters 16, 17 are removed therefrom.

In the illustrated embodiment, the first adapter 16 comprises a typical endotracheal connector configured to removably secure a manual resuscitation bag valve system 19 thereto. The first adapter 16 is configured to affix to the front side 13, such that forces exerted by a user to remove the manual resuscitation bag valve system 19 from the first adapter 16 does not remove the first adapter 16 from the plate 12. The first adapter 16 can comprise a variety of sizes to support various manual resuscitation bag valve systems 19.

The second adapter 17 is configured to removably secure a suction tubing 18 thereto via friction fit. The suction tubing 18 is secured over the second adapter 17 such that frictional forces between an interior wall of the suction tubing 18 and the second adapter 17 removably secure the suction tubing 18 thereto. In the illustrated embodiment, the second adapter 17 comprises a tapered shape allowing the second adapter to receive suction tubing 18 of various diameters thereon, however, in alternate embodiments, the second adapter 17 comprises a cylinder having a constant diameter.

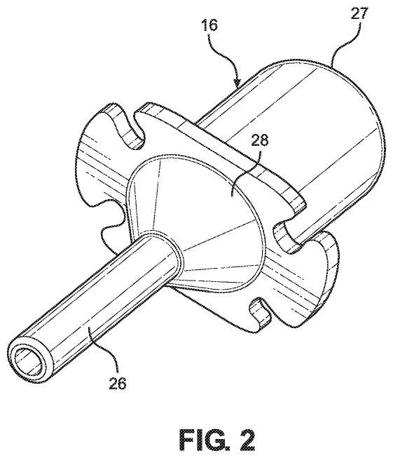

Referring now to FIG. 2, there is shown a perspective view of the first adapter of an embodiment of the airway support storage platform. In the illustrated embodiment, the first adapter 16 comprises a tubular member having a front end 27 opposite a rear end 28, wherein a stem 26 extends coaxially from the rear end 28. In the shown embodiments, the front end 27 and the rear end 28 comprise the same diameter such that the first adapter 16 comprises a consistent diameter therebetween. The manual resuscitation bag valve system is configured to removably secure to the front end 27 of the first adapter 16, such that the manual resuscitation bag valve system is readily available to a medical practitioner in the vicinity of a patient. The stem 26 is typically configured to secure within an endotracheal tube, however, in this system, the stem 26 removably secures within the plate to allow a storage location for the manual resuscitation bag valve system. In the illustrated embodiment, the stem 26 comprises a diameter less than that of the front and rear ends 27, 28. In some embodiments, planar projections extend orthogonally from the rear end 28, wherein the planar projections are configured to provide a gripping surface for a user to allow easy removal of the manual resuscitation bag valve system from the first adapter 16. In the illustrated embodiment, the planar projections are coplanar with the rear end 28 to allow the planar projections to rest flush against the plate when the first adapter 16 is secured thereto, thereby minimizing the risk of dislodging the first adapter 16 from the plate.

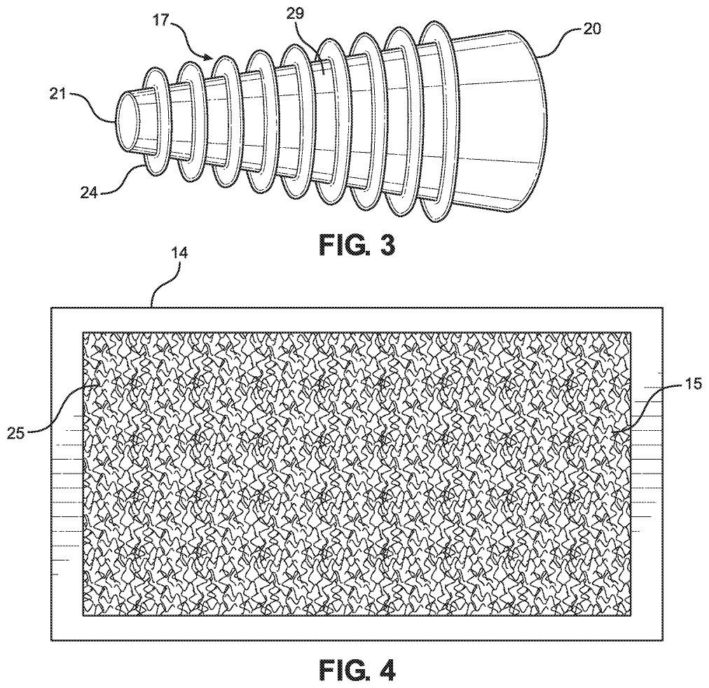

Referring now to FIG. 3, there is shown a perspective view of the second adapter of an embodiment of the airway support storage platform. The second adapter 17 comprises an exterior surface 29 configured to removably secure an interior wall of the suction tubing thereto via friction fit. In the illustrated embodiment, the second adapter 17 comprises a tapered shape, wherein a proximal end 20 of the second adapter 17 comprises a larger diameter than a distal end 21 of the second adapter 17. In this way, the user can easily secure suction tubing of various sizes to the second adapter 17, as the gradually increases in diameter from the distal end 21 towards the proximal end 20. In the shown embodiment, the second adapter further comprises a plurality of ridges 24 along the exterior surface 29 thereof. The plurality of ridges 24 are configured to provide increased frictional securement to the suction tubing, decreasing the risk of the suction tubing falling or otherwise being unintentionally disconnected from the second adapter 17, thus further limiting the risk of contamination of the suction tubing via unwanted contact with a floor surface. In the illustrated embodiment, the plurality of ridges 24 are concentric and spaced at regular intervals along the exterior surface. In other embodiments, the second adapter 17 further comprises a stem, such as that shown and described in relation to the first adapter in FIG. 3 above, wherein the stem is configured to removably secure the second adapter 17 to the plate.

Referring now to FIG. 4, there is shown a rear view of an embodiment of the airway support storage platform. A fastener 15 is affixed to the rear side 14 of the plate, wherein the fastener 15 is configured to removably secure the plate to a support surface, such as a wall. The fastener 15 can comprise a variety of different fasteners, such as adhesive, hook and loop, or the like. In embodiments wherein the fastener 15 comprises an adhesive, a backing layer can be removably securable to the adhesive to preserve the adhesive until a user desires to affix the plate to the support surface. In the illustrated embodiment, the fastener 15 comprises a hook and loop fastener configured to removably secure to a complementary fastener disposed on the support surface. In some embodiments, a perimeter of the fastener 15 and the rear side 14 are aligned, ensuring maximal securement to the support surface. In this way, the plate is prevented from being removed from the support surface when external forces are applied to remove the airway support tools, such as the manual resuscitation bag valve system and the suction tubing. However, in the illustrated embodiment, the cross-sectional area of the fastener 15 is less than that of the rear side 14, such that a lip 25 is formed therearound. The lip 25 is configured to provide a gripping surface to a user, allowing the user to easily remove the plate from the support surface when necessary.

In one exemplary use, the user removably secures the fastener 15 to the support surface adjacent to a patient's treatment area. The first and second adapters are secured to the plate and a manual resuscitation bag valve system and a suction tubing are removably secured to the first and second adapters, respectively. In this way, the airway support tools are readily available and easily accessible when needed, while being stored in a sanitary way. Furthermore, as each airway support storage platform can be easily secured adjacent to a patient's bedside, it further ensures that each airway support tool is only used for a single patient, minimizing risks of contamination.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. An airway support storage platform, comprising:
a plate having a front side opposite a rear side;
wherein a fastener is dispose don't he rear side, the fastener configured to removably secure the plate to a support surface;
a first adapter affixed to the front side;
wherein the first adapter comprises an endotracheal tube connector;
a second adapter affixed to the front side;
wherein the second adapter is configured to frictionally engage an interior wall of a suction tubing thereon.

2. The airway support storage platform of claim 1, wherein the second adapter tapers from a proximal end thereof towards a distal end thereof, such that the distal end comprises a diameter less than that of the proximal end.

3. The airway support storage platform of claim 1, wherein the first adapter is removably securable to the front side via a snap fit connection.

4. The airway support storage platform of claim 1, wherein the second adapter is removably securable to the front side via a snap fit connection.

5. The airway support storage platform of claim 1, wherein the fastener comprises a hook and loop fastener configured to removably secure to a complementary fastener disposed on the support surface.

6. The airway support storage platform of claim 1, wherein the fastener is disposed along an entirety of the rear side of the plat, such that a perimeter of the fastener and a perimeter of the base are aligned.

7. The airway support storage platform of claim 1, wherein an area of the fastener is less than that of the rear side, defining a lip about the rear side.

8. The airway support storage platform of claim 1, wherein the fastener comprises an adhesive.

9. An airway support storage platform, comprising:
a plate having a front side opposite a rear side;
wherein a fastener is dispose don't he rear side, the fastener configured to removably secure the plate to a support surface;
a first adapter affixed to the front side;
wherein the first adapter comprises an endotracheal tube connector;
a second adapter affixed to the front side;
wherein the second adapter further comprises a plurality of ridges disposed along an outer surface thereof, the plurality of ridges configured to frictionally engage an interior wall of a suction tubing thereon.

10. The airway support storage platform of claim 9, wherein the second adapter tapers from a proximal end thereof towards a distal end thereof, such that the distal end comprises a diameter less than that of the proximal end.

11. The airway support storage platform of claim 9, wherein the first adapter is removably securable to the front side via a snap fit connection.

12. The airway support storage platform of claim 9, wherein the second adapter is removably securable to the front side via a snap fit connection.

13. The airway support storage platform of claim 9, wherein the fastener comprises a hook and loop fastener configured to removably secure to a complementary fastener disposed on the support surface.

14. The airway support storage platform of claim 9, wherein the fastener is disposed along an entirety of the rear side of the plat, such that a perimeter of the fastener and a perimeter of the base are aligned.

15. The airway support storage platform of claim 9, wherein an area of the fastener is less than that of the rear side, defining a lip about the rear side.

16. The airway support storage platform of claim 9, wherein the fastener comprises an adhesive.

17. The airway support storage platform of claim 9, wherein the plurality of ridges are concentric.
